(19) United States
(12) Patent Application Publication
(10) Pub. No.: US 2026/0237227 A1
(43) Pub. Date: Aug. 13, 2026

(54) SYSTEMS AND METHODS FOR MICROORGANISM IDENTIFICATION

(71) Applicant: Drizzle Health LLC, Baltimore, MD (US)

(72) Inventors: Bonolo Mathekga, Baltimore, MD (US); Yukari Manabe, Elkridge, MD (US); Digvijay Singh, Baltimore, MD (US)

(21) Appl. No.: 18/866,403

(22) PCT Filed: May 17, 2023
(86) PCT No.: PCT/US2023/067150

§ 371 Date: May 16, 2025

Related U.S. Application Data
(60) Provisional application No. 63/343,462, filed on May 18, 2022.
(60) Provisional application No. 63/343,459, filed on May 18, 2022.

(51) Int. Cl. G06V 20/69; G01N 21/00; G01N 21/01

(52) U.S. Cl. CPC G06V 20/69; G01N 21/00; G01N 2021/0181

(57) ABSTRACT

This disclosure discusses a Mueller Matrix Polarimetry system for real time identification of microorganisms in the field. The disclosed systems comprise a mechanical arrangement of optical elements that are configured to create a library of characteristic measurements for each organism. This library acts as a reference for comparison for signatures measured from unknown samples in the field. Elements described in the disclosure minimize moving parts as well as manual intervention, lending to use in portable, battery-operated devices.

### CROSS-REFERENCE TO RELATED APPLICATIONS

This application claims the priority benefit of U.S. Provisional Patent Application Ser. No. 63/343,459, filed on May 18, 2022; and U.S. Provisional Patent Application Ser. No. 63/343,462, filed on May 18, 2022, the contents of each of which are herein incorporated by reference in their entireties.

### INCORPORATION BY REFERENCE

All publications and patent applications mentioned in this specification are herein incorporated by reference in their entirety, as if each individual publication or patent application was specifically and

### TECHNICAL FIELD

This disclosure relates generally to the field of biotechnology, and more specifically to the field of diagnostics. Described herein are systems and methods for sample identification.

### BACKGROUND

Access to diagnostic tests is essential to the delivery of quality healthcare. The Lancet commission found that approximately 47% of the global population has little to no access to diagnostics. For example, there are an estimated 1.1 million premature deaths annually in low-income and middle-income countries (LMICs) that could be avoided by improving diagnostics for at least the following conditions: diabetes, hypertension, Human Immunodeficiency Virus, hepatitis B virus infection, and syphilis.

For diagnostic tests to be effective in LMICs, diagnostic tests should easily fit into existing workflows and utilize infrastructure that is already available. The diagnostic tests should be adaptable to inconsistent electricity supplies, dust, humidity, and uncontrolled temperatures. Diagnostic tests routinely used in and designed for high-income countries are imported into LMICs and expected to yield the same positive outcomes, which is impractical and unlikely.

Sputum Smear Microscopy (SSM) is a widely used test for diagnosing tuberculosis (TB), with more than 77.6 million tests performed per year. Despite having the low sensitivity as compared to other diagnostic tests (sensitivity of ~50%), it is still a widely utilized diagnostic test.

In recent years, Nucleic Acid Amplification Tests (NAAT) have been introduced to National TB programs. Despite World Health Organization (WHO) recommendations and increased funding, the uptake and accessibility of these tests has been low. This is because these machines ($30k) and assays are high cost (~$15/test) and require sophisticated infrastructure that is not available at the primary health centers (PHC) where TB patients access the system. In India, for example, NAAT based tests are limited to tertiary health centers, inaccessible to patients in terms of (1) location-these centers are based in cities (a city usually has one or two of these high-cost systems), and (2) patients typically have to be referred from a primary health center before getting a NAAT test at the district level facilities. In contrast, in villages, patients will typically go through village centers and subcenters, before reaching primary health centers where they can access SSM if referred.

Community Care Workers (CCWs), for example, decentralize TB services by reaching patients at both PHC clinics and at their homes. CCWs in India, for example, routinely cover a hundred people per day. A device in the hands of CCWs could enable TB programs to move at the pace of the disease and its transmission. It will enable real-time surveillance, surgical surge of resources, localized quarantines and care in communities often left out of the system.

Accordingly, new devices, systems, and methods are needed to diagnose infections accurately and quickly, especially in underserved populations.

### SUMMARY

In some aspects, the techniques described herein relate to a portable system for detecting one or more microorganisms in a specimen, including: one or more light sources arranged to produce a light beam that is configured to interact with a receptacle positioned in the system; one or more filters, wherein the light beam is polarized by at least one of the one or more filters; one or more beam splitters; a plurality of sensors arranged along an arc, relative to a path of the light beam, and configured to detect scattered light at a plurality of angles relative to the receptacle; and a processor communicatively coupled to memory, the one or more light sources, and the plurality of sensors, the processor configured to execute instructions stored in the memory, the instructions including: receiving, from at least a subset of the plurality of sensors, detected scattered light signals from a sample that is positionable in the receptacle; identifying one or more optical signatures in the detected scatter signals from the sample, comparing the one or more identified optical signatures from the sample to a database of optical signatures, and outputting, based on the comparison, an indication of the one or more identified optical signatures from the sample.

In some aspects, the techniques described herein relate to a portable system for detecting one or more microorganisms in a specimen, the system including: a light source arranged to produce a light beam that is configured to interact with a receptacle positioned in the system; a polarization state generator configured to polarize the light beam from the light source; a polarization state analyzer configured to receive the polarized light beam, the polarized light beam having interacted with a sample that is positionable in the receptacle, and determine one or both of: one or more Mueller matrix parameters or one or more Stokes four-vectors parameters of the polarized light beam; and one or more processors communicatively coupled to memory, the light source, the polarization generator, and the polarization state analyzer, the processor configured to receive instructions from the memory and execute the instructions including: receiving one or more determined parameters from the polarization state analyzer, the sample positionable in the receptacle, identifying one or more optical signatures of the sample based on the one or more determined parameters, comparing the one or more identified optical signatures of the sample to a database of signatures, and outputting an indication of the one or more identified optical signatures of the sample.

In some aspects, the techniques described herein relate to a computer-implemented method of identifying one or more microorganisms in a specimen, including: receiving detected scattered light signals, from one or more sensors, from a sample that is positionable in a receptacle, the sample having interacted with a polarized light beam; identifying one or more optical signatures in the detected scatter signals from the sample, comparing the one or more identified optical signatures from the sample to a database of optical signatures, and outputting, based on the comparison, an indication of the one or more identified optical signatures from the sample.

In some aspects, the techniques described herein relate to a non-transitory computer-readable storage medium for use in conjunction with a computer, the computer-readable storage medium storing programs instructions that, when executed by the computer, cause the computer to carry out one or more operations including: receiving detected scattered light signals, from one or more sensors, from a sample that is positionable in a receptacle, the sample having interacted with a polarized light beam; identifying one or more optical signatures in the detected scatter signals from the sample, comparing the one or more identified optical signatures from the sample to a database of optical signatures, and outputting, based on the comparison, an indication of the one or more identified optical signatures from the sample.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 shows a schematic of an embodiment of a system for sample identification.

FIG. 2 shows a schematic of an embodiment of a system for sample identification.

FIG. 3 shows a schematic of an embodiment of a system for sample identification.

FIG. 4 shows a schematic of an embodiment of a system for sample identification.

FIG. 5 shows a schematic of an embodiment of the system for sample identification.

FIG. 6 shows a schematic of another embodiment of a system for sample identification and including moving parts.

FIG. 7 shows a schematic of another embodiment of a system for sample identification and including moving parts.

FIG. 8 shows a schematic of an embodiment of a system using mirrors arranged along an ellipse and including no moving parts.

FIG. 9 shows a schematic of an embodiment of a system including no moving parts and a detector in proximity to the sample.

FIG. 10 shows a schematic of an embodiment of a method for sample identification.

FIG. 11 shows a flow chart of an embodiment of a method for sample identification.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated embodiment(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Described herein are systems and methods for detecting bacteria in samples, such as sputum and saliva, with high accuracy, a turnaround-time of about a minute, a small footprint, and a lower cost.

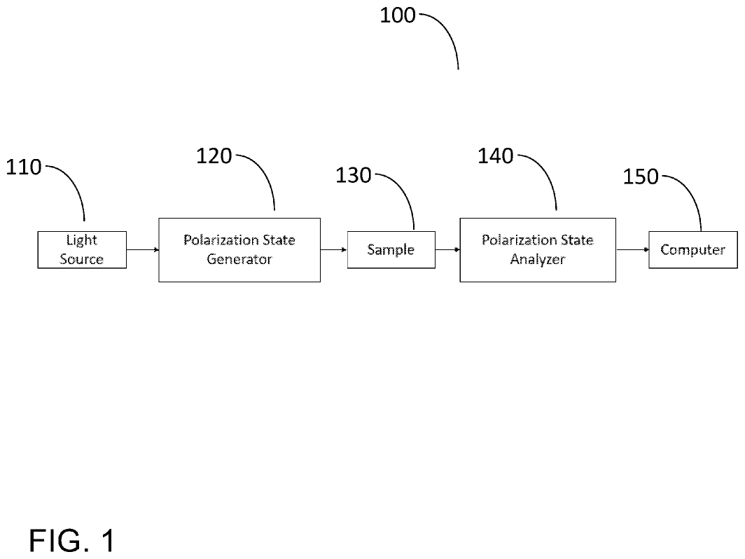

In general, the systems and methods described herein follow the Free Space Optics paradigm (i.e., all the light passes through air to scatter, interact, etc.). In some embodiments, similar arrangements, as those shown in FIGS. 1-9, can be employed on a chip (but with much smaller components) in integrated photonics paradigm, fiber optics communications, etc.

Described herein are systems, devices, and methods for testing specimens and identifying microorganisms, bacteria, viruses, etc. in the specimens. In some embodiments, the systems, devices, and methods can include, but are not limited to, optical sensors, beam splitters, filters, light polarizers (vertical, horizontal, left, and right), lasers, microcontroller, and a computer system for signal processing and results output.

Described herein are optical reader-based systems and methods to detect and identify species, components, materials, etc. based on a phenotypic signature that is specific to that particular species, component, material, etc. In some embodiments, the systems and methods described here may be used to detect and identify bacterial species. Various aspects of the systems and methods described herein use a library of signatures such as those for M. Smegmatis, Nocardia, E. coli, and Staphylococcus Aureus, to enable detection of various strains. In some aspects, the devices and methods described herein can be used for door-to-door screening of infectious diseases, like Mycobacteria tuberculosis, by community healthcare workers in hard to reach or poor communities and clinics.

Described herein are systems, devices, and methods for phenotypic identification of microorganisms, viruses, etc. based on a predetermined signature. For example, in non-limiting embodiments, the systems, devices, and methods described herein can be used to identify Tuberculosis, Staphylococcus Aureus, Escherichia Coli, Nocardia, etc. in specimens. The systems, devices, and methods described herein are configured to identify phenotypic signatures of microorganisms contained within a specimen or sample (e.g., sputum, blood, saliva, serum, urine, stool, buccal swabs, nasal swabs, throat swab, etc.). It may be noted that the use of the devices and systems described herein may be expanded to include a variety of sample types, including but not limited to, in vitro human samples (e.g., sputum, urine, saliva, etc.), water or food samples for consumption related purposes, chemical solutions for analysis in research and engineering contexts, and other electronic diagnostics contexts.

Further, various embodiments described herein may additionally, or alternatively, be used for material science characterization and/or research, for example thin film characterization. In some embodiments, any of the systems (e.g., FIGS. 1-9) may be used to assess (e.g., determine thickness, optical constant, refractive index, etc.) a barrier layer during and/or after packaging deposition on or around a food product. For example, a positioning or location of one or more sensors and/or one or more mirrors in any of the systems of FIGS. 1-9 may be adjusted for assessment of a barrier layer.

The technical problem sought to be solved was decentralizing microorganism testing (e.g., bringing testing closer to the point of care) by designing, simplifying, miniaturizing, and making the testing apparatus and methods to provide actionable results, portable and nearly instantaneous. This may lead to faster treatment initiation, and consequently reduce the ongoing transmission of diseases in communities. The technical solution provided by the systems, devices, and methods described herein include a portable (e.g., handheld) device that can detect and identify the organism present in a sample by querying an existing library of signatures and looking for a match.

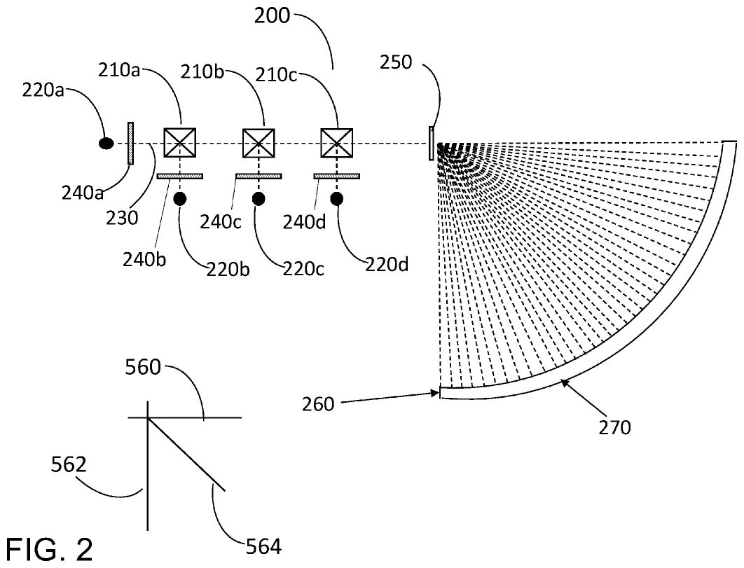

In some embodiments, the components (e.g., any of the components of FIGS. 1-9) within a portable or handheld device may be arranged such that the portable device is easily transportable. For example, in some embodiments, the dimensions of a portable device may be about 12 inches (30.48 cm) by 18 inches (45.72 cm) by 6 inches (15.24 cm) to about 8 inches (20.32 cm) by 4 inches (10.16 cm) by 4 inches (10.16 cm). In some embodiments, the dimensions of a portable device may be less than about 8 inches (20.32 cm) by 4 inches (10.16 cm) by 4 inches (10.16 cm). In some embodiments, for example as shown in FIG. 2, a distance between a first beam splitter and a second beam splitter may be about 0.5 inches (1.27 cm) to about 3 inches (7.62 cm); about 1 inch (2.54 cm) to about 2 inches (5.08 cm); about 1.5 inches (3.81 cm) to about 2.5 inches (6.35 cm); etc. In some embodiments, a distance between a beam splitter and a light source may be about 0.5 inches (1.27 cm) to about 4 inches (10.16 cm); about 1 inch (2.54 cm) to about 3.5 inches (8.89 cm); about 0.5 inches (1.27 cm) to about 1.5 inches (3.81 cm); etc. In some embodiments, the dimensions of a portable or handheld device may be reduced by adjusting a location or position of one or more sensors, detectors, or mirrors.

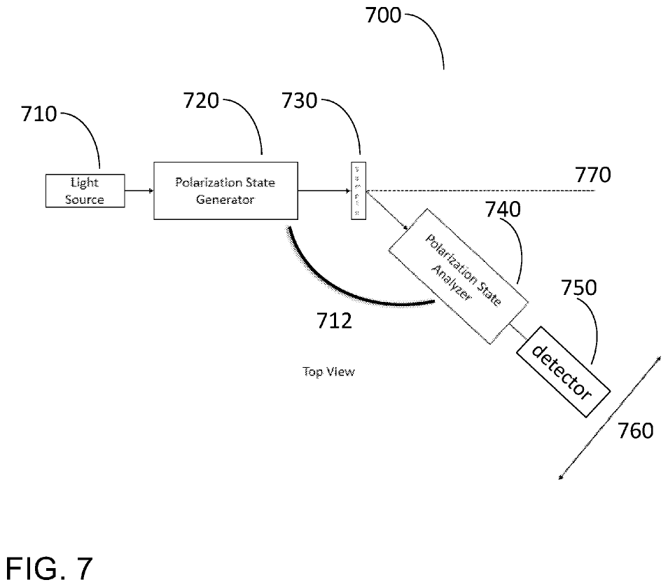

In an embodiment, the system may function as follows: The sample to be investigated is collected and smeared on a receptacle (e.g., glass slide, plastic slide, or the like). The system is calibrated using a blank or clean receptacle (i.e., the light scattering properties of a clean or blank receptacle are recorded). The sample under investigation is inserted into the system, and the optical properties of the sample are recorded by the sensors located at different angles, while different light configurations are passed through it. The polarized light configurations include but are not limited to: horizontal linear (H), vertical linear (V), right (R) (i.e., a clockwise rotation corresponds to a right-hand circular polarization state and a phase shift of −π1/2), and left (L) (i.e., a counterclockwise rotation refers to left-hand circular polarization state and a phase shift of +π/2). The detected light intensities at various angles are read by a microcontroller (optionally including an analog to digital converter, digital signal processor, etc.). The digital data generated by the light intensity analysis is described in connection with FIG. 7.

Research in the description of polarized light has progressed in the last two decades. This has been realized in the use of polarimeters and ellipsometers for non-destructive investigation of materials, thin films, and surfaces. This has centered around Mueller matrices and Stokes four-vectors, describing states of incident and scattered light from a scatterer. Intensity and/or degree of scatter can depend on the number of scatterers, shape, size, refractive index, diffraction properties, autofluorescence, direction specific forward scattering related to complexity of cell structures, etc.

The Mueller matrix and Stokes four-vectors are mathematical representations used to describe the polarization state of a light beam. The Mueller matrix is a 4×4 matrix that relates the input and output polarization states of a beam of light through linear transformations. It describes the polarization properties of an optical system, including transmission, reflection, and scattering characteristics. The Mueller matrix is denoted by M and relates the input Stokes vector (S_in) to the output Stokes vector (S_out) as follows: S_out=M*S in.

S_in and S_out are 4-element column vectors known as Stokes vectors. The elements of the Stokes vector represent the intensities and polarizations of the light in different orthogonal states. The Mueller matrix, M, contains 16 elements that quantify the transformation of the input polarization state to the output polarization state.

The Stokes vector is a four-element vector used to describe the polarization state of a beam of light. It characterizes the intensity and polarization properties of the light in terms of four parameters: S0, S1, S2, and S3.

The Stokes vector is given by: S=[S0, S1, S2, S3]
S0 represents the total intensity of the light.
S1 and S2 represent the linear polarization components along two orthogonal axes.
S3 represents the circular polarization component.

The Stokes vector describes the polarization state of a beam of light and can be used to calculate various polarization parameters, such as degree of polarization, polarization ellipse, and polarization angle.

Taken together, the Mueller matrix describes the transformation of the input polarization state to the output polarization state of a light beam through a linear optical system, while the Stokes vector represents the polarization state of the light using four parameters: S0, S1, S2, and S3.

Previous devices measuring Mueller matrices used reflected light. Preliminary research has investigated use of polarized light in imaging tissues as well as cellular solutions for diagnostic purposes. These have generally been non-portable devices with multiple moving parts.

The technical problem sought to be solved with the systems, devices, and methods described herein was how to analyze specimens or samples in the field by healthcare workers in door-to-door or peripheral settings apart from usual laboratory settings. Further, use of Mueller Matrix polarimetry has not been investigated for use in infectious disease diagnostic contexts, such as those involving Escherichia coli, Mycobacterium Tuberculosis, etc. In some aspects, the systems described herein include a hardware component that generates and reads polarized light signals, and computer-readable instructions that include performing data analysis algorithms and generating one or more diagnostic results outputs. In some implementations, the computer-readable instructions may be stored in memory on a remote server (e.g., via internet or Cloud access) and executed by one or more processors of the server. In some implementations, the computer-readable instructions may be stored in memory (e.g., as firmware) in a handheld or portable device and executed by one or more processors in the handheld or portable device. Such a configuration may be useful for diagnostic testing in areas and/or communities where high-speed internet connectivity is problematic. In still further implementations, the computer-readable instructions may, in part, be stored in memory on a remote server and, in part, stored in memory in a handheld or portable device and executed by one or more processors of the server and/or the handheld or portable device. For example, the execution of the computer-readable instructions may be distributed across one or more devices, remote or local.

Further, the systems described herein use light transmitted through an optically transparent receptacle or sample holder. In an embodiment, this is a plastic slide with a sample deposited thereon. In another embodiment, it can be a glass slide. In another embodiment, it can be a microfluidic chip. In some embodiments, the sample may be a completely solid sample, reducing biosafety risks as well as problems with handling liquid samples.

In general, various features of the systems, devices, and methods described herein may include mechanical systems configured to use a slide instead of a solution. The systems described herein may include no moving parts. The systems described herein may use transmitted light rather than reflected light. In some aspects, the systems described herein may be portable systems and devices. The systems, devices, and methods described herein may use and/or generate: a Mueller matrix used for sample identification; software-based analysis to identify and/or determine patterns or signatures specific to one or more microorganisms; and/or a database or library of patterns or signatures.

In general, the various features of the systems, devices, and methods described herein, when optionally combined with sample preparation devices described elsewhere herein, can provide increased sensitivity for identifying specific bacteria.

In any of the embodiments described herein, for example any of FIGS. 1-9, one or more sensors may be arranged at least partially around a circumference or partial circumference of a sphere with its center at the light scattering site (e.g., the point at which the light source interacts the sample). In some embodiments, the various embodiments (e.g., FIGS. 1-9) described herein function detect an intensity of light at various angles and/or an intensity of scattered light that has a certain polarization at various angles ..

FIG. 1 shows a schematic of an embodiment of a system 100 for sample identification including no moving parts. The system 100 can include the following components listed according to the flow of light: a monochromatic light source 110 (optionally a laser) that is selected for its wavelength and power characteristics to appropriately provide sensitivity and signal for a given species of bacteria; a polarization state generator 120 (also herein called a polarizer), and optionally, an optically transparent conduit or receptacle 130 to hold (e.g., contain, receive therein or thereon) a sample (such as a plastic or glass slide, or other such materials). The receptacle 130 may include, but is not limited to, a microscopy slide, a polymer sheet, glass, etc. The light scattered from the sample is passed through a polarization state analyzer 140 (optionally, a rotating analyzer and/or compensator). The light signal is detected by a detector (e.g., light sensor) and converted to digital signals (e.g., through an analog to digital converter, ADC) and input into a computing device 150 where it can be analyzed using one or more processors configured to execute instructions for identifying and/or determining a signature and/or pattern of a microorganism for a particular sample received at the receptacle 130.

In some embodiments, a light source 110 can be selected based on the bacterial species of interest. For example, a light source 110 that emits substantially about a wavelength of about 532 nm and/or about 633 nm may enable detection of a wide variety of bacteria including, but not limited to Mycobacteria, Staphylococcus species, Nocardia, Escherichia species (e.g., E. coli), and Salmonella, Cholera species (e.g., Vibrio). In another example, a light source 110 that emits substantially at a wavelength of about 450 nm, about 785 nm, and/or about 850 nm may enable detection of bacteria such as Mycobacteria.

In general, any of the polarization state generators described herein may receive light from the light source 110 and in response, may generate different polarization states (i.e., H, V, R, L etc.). The polarized light may be received at the sample. Polarization state generators and analyzers can include photo elastic modulators, combinations of rotating polarizers, and/or variable incident signals. Use of liquid crystal-controlled elements may allow switching generators and analyzers electronically, thereby reducing mechanical manipulation in the system. This enables improved measurement accuracy and measurement speed as compared to conventional sample analysis systems.

In general, any of the polarization state generators and/or the polarization state analyzers described herein optionally include one or more linear polarizers, one or more circular polarizers, and/or one or more waveplates collinearly placed in a sequence. In general, a linear polarizer may transmit substantially uniform light vibrating in a first plane while absorbing in a second plane that is orthogonal to the first plane. In general, a circular polarizer is a linear polarized filter and a quarter-wave plate.

FIG. 1 shows a generalized system 100 for sample identification. FIGS. 2-6 show various implementations of the system 100 of FIG. 1. FIG. 2 shows a system 200 for sample identification with no moving parts. The system 200 can include one or more filters 240, one or more lasers 220, one or more beam splitters 210, and one or more sensors 260. In some embodiments, system 100 includes a light source, a filter, a beam splitter, and a sensor. In some embodiments, system 100 includes a plurality of light sources, a plurality of filters, a plurality of beam splitters, and a plurality of sensors. For example, there may be one, two, three, four, one to five, five to ten, ten to fifteen, fifteen to twenty, twenty to fifty, fifty to 100, etc. light sources, filters, beam splitters, and/or sensors. In some embodiments, any of the one or more sensors may be covered with a polarization film in various directions. In some embodiments with a plurality of sensors, there may be about one row to about 10 rows, for example 4 rows or 5 rows of about 50 sensors, collectively, on the surface of a sphere. In some embodiments, four rows may function to measure an intensity of scattered light polarized in certain ways, and a fifth row may function to measure an intensity (in an unpolarized state). In some embodiments, there may be a row of one or more sensors having H polarization, a row of one or more sensors having a P polarization, a row of one or more sensors having an M polarization, and a row of one or more sensors having a V polarization. In some embodiments, the one or more sensors may be photodetectors, charge-coupled device, or other electronic detectors.

For ease of understanding, longitudinal axis 560 is the x-axis (and light path for light source 220a), axis 562 is the y-axis; and axis 564 is the z-axis. The following components of system 200 are described along the light path 230 (along axis 560): light source 220a, filter 240a, and beam splitter 210a. In some embodiments, light path 230 includes one or more beam splitters or a plurality of beam splitters. In some embodiments, light path 230 also receives light from laser 220b that passes through filter 240b (laser 220b and filter 240b being orthogonal to axis 560) and beam splitter 210a. In some embodiments, light path 230 also receives light from laser 220c that passes through filter 240c (laser 220c and filter 240c being orthogonal to axis 560) and beam splitter 210b. In some embodiments, light path 230 also receive light from laser 220d that passes through filter 240d (laser 220d and filter 240d being orthogonal to axis 560) and beam splitter 210c. The light path 230 interacts with an optional receptacle 250 for retaining a sample. One or more optical sensors 260 can be placed along an arc 270 (in an x-y plane) to detect scattered light at various angles. This can improve stability of vibration prone system components and reduces time for reading signals and generating results. This in turn is beneficial for increasing the turnaround rate of test results/sample identification results (i.e., within minutes) for high volumes, such as those in a clinical lab, or turnaround rate of test results/sample identification results (i.e., within seconds) for low volumes, such as those in a community-based setting. Such a static system in turn allows itself to be sealed from moisture and temperature related perturbations that may affect component and device performance.

In some embodiments, to seal a system (any of the systems of FIGS. 1-9) from moisture and/or temperature related perturbations, the system may be nitrogen purged the after each use. In some embodiments, one or more desiccants (e.g., silica gel, activated charcoal, calcium chloride, charcoal sulfate, activated alumina, montmorillonite clay, molecular sieve, etc.) may be used in the system. In some embodiments, the system may have predefined constraints using a predefined dewpoint (e.g., shouldn't use the system outside of a temperature range of about 10 Celsius to about 40 Celsius) based on a determined pressure of the system. In some embodiments, the system can include one or more humidity sensors to monitor a humidity of the system and alert a user when humidity around or in the device is outside a predefined range, suggesting that the system should not be used. In some embodiments, the systems described herein may be sealed, except for a slot where the receptacle is received into the system. In some embodiments, the slot may be sealed with a flap, door, and the like when the system is not used or when the system is actively processing a sample.

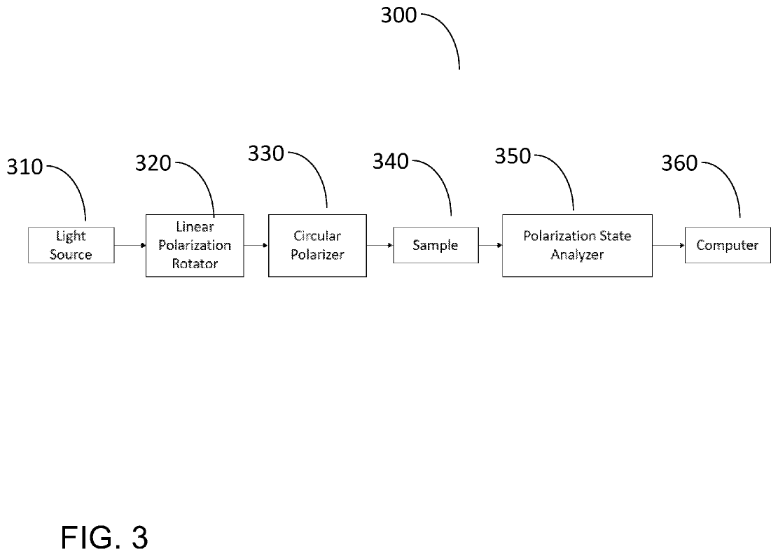

As shown in FIG. 3, an embodiment of a system 300 for sample identification may include: a light source 310, a linear polarization rotator 320, a circular polarizer 330, optionally a sample conduit or receptacle 340 for containing a sample, and a polarization state analyzer 350. System 300 may be configured to be communicatively coupled to a computing device 360. In some embodiments, linear polarization rotator 320 and/or circular polarizer 330 can be replaced with electronically actuated components such as birefringent quartz crystals, for example to reduce the size of the system, enabling, for example, community care workers to transport the system and/or devices.

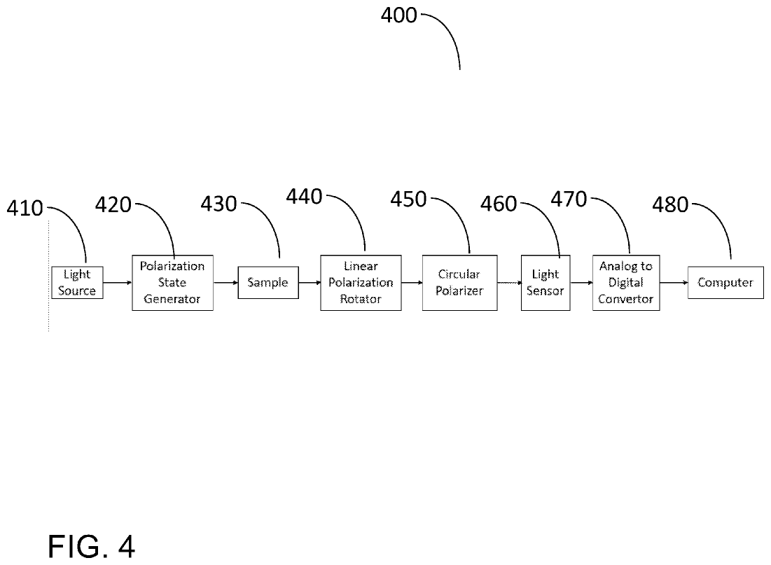

As shown in FIG. 4, an embodiment of a system 400 for sample identification may include: a light source 410, a polarization state generator 420, optionally a sample conduit or receptacle 430 for containing a sample, a linear polarization rotator 440, a circular polarizer 450, a light sensor 460, and an analog to digital converter (ADC) 470. System 400 may be configured to be communicatively coupled to a computing device 480. In some embodiments, circular polarizer 450 can be replaced with electronically actuated components such as birefringent quartz crystals, for example to reduce the size of the system, enabling, for example, community care workers to transport the system and/or devices.

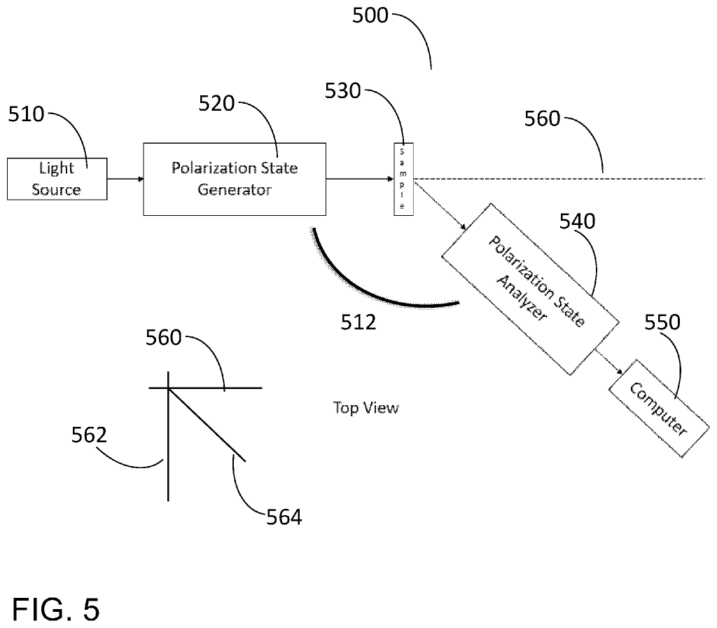

Turning now to FIG. 5, which shows a top view of the embodiment of the system of FIG. 2. As shown in FIG. 5, system components may be arranged relative to the receptacle 530; and parameters at various angles to incident light can be measured. For example, the system 500 of FIG. 5 uses forward scattering in the Mie scattering paradigm. The detector or the polarization state analyzer 540 can measure light scattered at an angle 512 (in an x-y plane) from the receptacle 530. For ease of understanding, longitudinal axis 560 is the x-axis, axis 562 is the y-axis; and axis 564 is the z-axis. The angle 512 in the x-y plane may be about 0 degrees to about 90 degrees; 10 degrees to about 40 degrees; about 90 degrees to about 180 degrees; about 90 degrees to about 120 degrees, about 60 degrees to about 120 degrees; about 110 degrees to about 160 degrees; etc. For example, a light source 510, a polarization state generator 520, and an optional conduit or receptacle 530 for receiving a sample may be substantially linearly aligned along longitudinal axis 560 while a polarization state analyzer 540 may be offset relative to an x axis or longitudinal axis 560. Optional computing device 550 can be communicatively coupled to the system 500 and may be similarly offset relative to longitudinal axis 560 or located at a remote location.

Alternatively, in any of the embodiments described herein, measurement at various angles can accomplished by rotation of the detection device (e.g., Polarization state analyzer) using a stepper motor controlled by a microcontroller, for example. Alternatively, as shown in various Figures herein (e.g., FIGS. 1, 2, 5, 8-9), a plurality of detectors may be used such that the device or system does not include moving parts (e.g., does not include a rotatable detector). Alternatively, as shown in various Figures herein, one or more mirrors can be used to reflect light into a detector, for example saving on cost and electronic complexity.

Any of the computing devices 150, 360, 480, 550, etc. described herein may be local computing devices (coupled to the system) or remote computing devices (e.g., server, workstation, etc.).

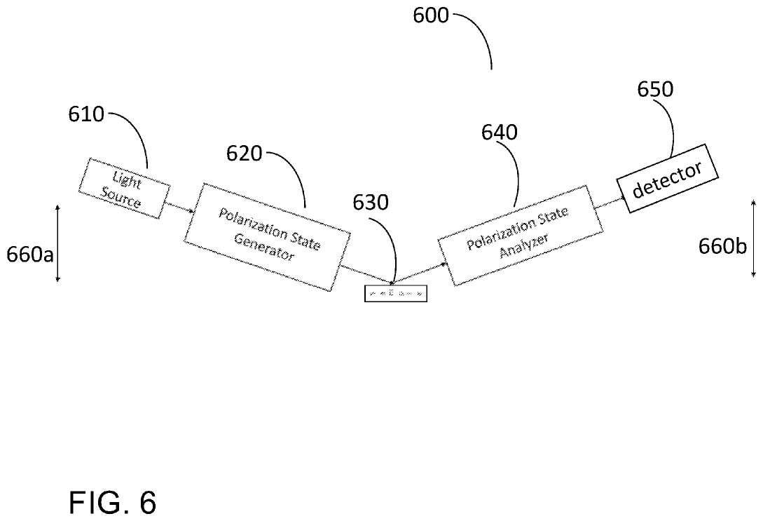

Turning now to FIGS. 6-9, which include additional configurations for Mueller Matrix imaging using ellipsometers. Ellipsometers generally measure reflected light and include a light source and detector. The implementations of FIGS. 6-9 are contrasted to that of FIGS. 1-5, which use polarization of light to measure scattered light polarizations. In general, for FIGS. 6-9, a detector can reside at a focus of one or more elliptical mirrors (the other focus can have the scattering sample). In some embodiments, light is incident and detected on a same side of the receptacle. In some embodiments, light is incident on a first side and detected on a second side of the receptacle. Light is scattered (forward or back scattered) and then reflected off the one or more mirrors, residing on an ellipse back into the detector. The geometric arrangement reduces the likelihood of having to use a plurality of detectors or moving parts. In some embodiments, the detector of FIGS. 6-8 includes a wide aperture.

As shown in FIGS. 6-8, a light source can emit light obliquely at a receptacle 630 in an arrangement. FIG. 6 includes similar components as FIG. 2, for example a light source 610, polarization state generator 620, optional sample receptacle 630, polarization state analyzer 640, and detector 650 (may optionally be integrated into a computer or other processor). The light source 610 and detector 650 can be manipulated up and down (e.g., via an actuator), according to arrows 660a, 660b, introducing a moving component. The movement of the light source 610 and detector 650 may be determined by a computing device communicatively coupled to system 600, for example based on collecting various angles of reflection and inputting the collected angles into a model. In some embodiments, the model can employ Snell's law for determining the movement of the light source and/or detector 650. This limits the speed of the system 600, increases maintenance, increases power requirements, increases complexity, and reduces the overall life of the system.

Another setup for a Mueller matrix-based device or system (which measures transmission but also optionally reflected light) is as shown in FIG. 7. FIG. 7 is similar to FIG. 5, except that the double-sided arrow indicated by 760 shows bidirectional movement of the detector 750. As in FIG. 5, the embodiment of FIG. 7 includes a light source 710, a polarization state generator 720, and optionally a sample receptacle 730 substantially linearly aligned along plane 770 while a polarization state analyzer 740 and detector 750 (e.g., sensor, computing device, etc.) may be offset relative to plane 770. Angle 712 may be adjusted according to the movement of detector 750.

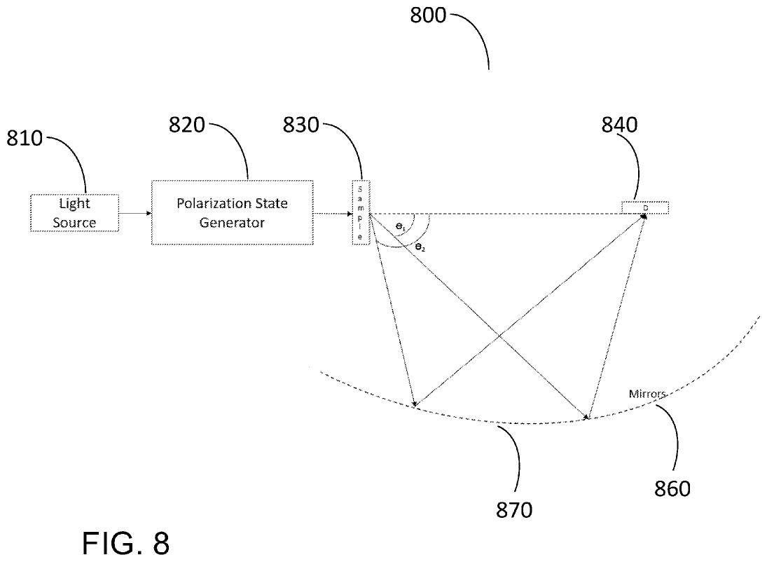

FIG. 8 shows another embodiment of a system 800 for sample identification. The dashed line shows an array of electronically controlled mirrors 860, laid along an ellipse 870 with known parameters. For example, the parameters may include the form: x\hat{ }2/a\hat{ }2+y\hat{ }2/b\hat{ }2=1, where a and b are the major and minor axis of the ellipse 870. The array of electronically controlled mirrors 860, in an embodiment, may include liquid crystal-controlled mirrors that can reflect and transmit based on electrical signals. The sample receptacle 830 and the detector 840 can be placed at two foci of the ellipse, so that light from a first focus (e.g., the sample receptacle 830), reflected from the ellipse 870, can interact with a second focus (e.g., where the detector 850 is located) according to the ellipse-focal theorem. In some embodiments, the array of mirrors 860 can be initially deactivated and sequentially activated, so that light scattered at an angle or various angles can be detected sequentially, without the risk of mixing multiple angles. Further, as shown in FIG. 8, system 800 is free from mechanical parts, leading to increased rates of measurement and angle switching.

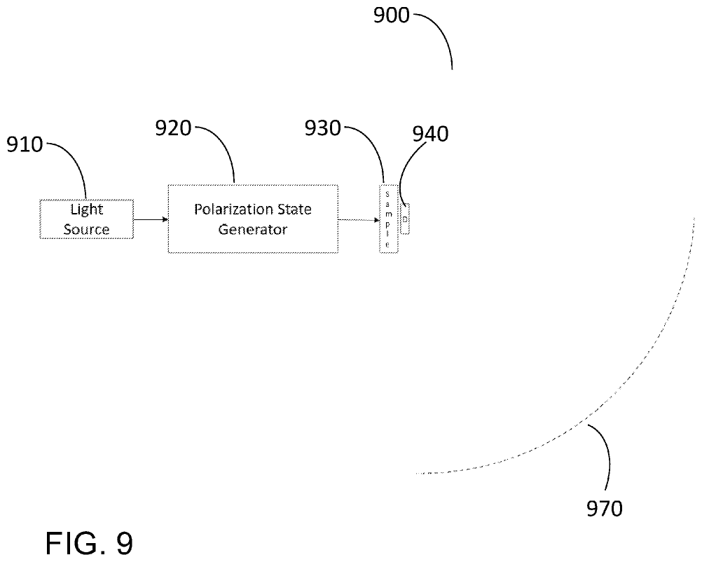

FIG. 9 shows another embodiment of a system 900 for sample identification. The arrangement of components in system 900 may be used, for example in handheld or portable systems that are space constrained and/or approximating a square shape in dimensions. For example, in some embodiments, the detector 940 can be moved closer to the sample receptacle 930 (downstream of the light source 910 and polarization state generator 920), leading to an ellipse 970 that approximates a circular shape (a circle is an ellipse with equal axes). In such an arrangement, the detector 940 is positioned such that the light scatter from the receptacle 930 can be collected by the detector 940, for example to capture at least about 0 degrees to about 20 degrees of scatter. The system 900 of FIG. 9 can be helpful in space conservation and optionally with detectors with narrow apertures. The detector 940 is close to the sample receptacle 930 and positioned in a manner to not obstruct a light path transmitted through the receptacle 930.

In some implementations, any of systems 100, 200, 300, 400, 500, 600, 700, 800, 900 may be calibrated. Calibration may include reading in a blank or empty or clean sample receptacle to generate a signal for the blank or empty or clean receptacle (i.e., noise). This calibration signal is subtracted from the signal for the sample of interest in the receptacle, resulting in a calibrated or cleaned signal of interest. The calibrated or cleaned signal of interest is further processed and analyzed to determine one or more microorganisms in the sample.

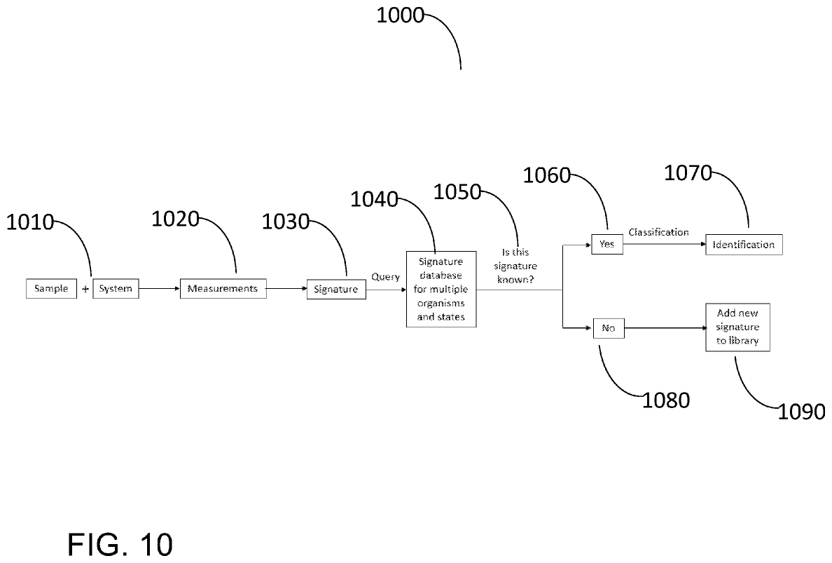

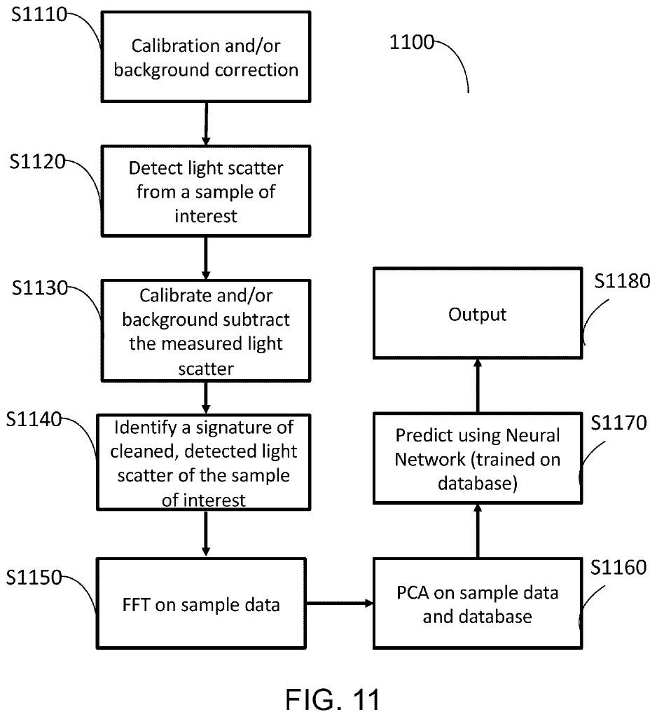

Turning now to FIGS. 10-11. Any of the algorithms described herein may be configured to receive, as an input, digitized signals indicative of scattered light (forward scattered or back scattered) from a sample responsive to illumination, process the signals, identify a pattern in the processed signals, and output an indication of a type of sample based on the identified pattern. For example, the type of sample may include, but is not limited to: a chemical, a microorganism, a material, etc. The pattern can be indicative of a sample type and/or indicative of one or more material or live components within a sample since a material, biological component, chemical, microorganism, etc. may scatter the light in a reproducible pattern. As used herein, a signature may be described as an output of an algorithmic processing of scattered light captured by any of the systems 100, 200, 300, 400, 500, 600, 700, 800, 900 described herein, such that the given arrangements of components as used in any of the systems can produce reproducible, consistent, and/or characteristic signatures coming from a species of scatterer (e.g., microorganism, chemicals, material, etc.). These recurring identifiable elements of the signature may be discernable regardless of concentration. Noise from interfering species of scatterers that may be present in a sample may be removed using signal processing algorithms (e.g., using time gating or the like) to recover the same signature. This can be achieved both mechanistically and by the way of digital signal processing.

In any of the embodiments of FIGS. 1-9, a sample may be purified, concentrated, or otherwise processed for imaging in any of the systems of FIGS. 1-9. One way to purify, concentrate, or otherwise process the sample is to use polymer technology where samples are centrifuged onto a sample type specific surface (e.g., bacterial species-specific surface, chemical specific surface, etc.) to collect nearly pure concentrations of the specific sample, species, chemical, etc. of interest. For example, a bodily fluid sample may be concentrated on a polymer that is specific for Mycobacteria, such that nearly pure concentrations of Mycobacteria are achieved. Another mechanism includes dead-end filtration or cross-flow filtration to remove debris and/or contaminants above a predefined size from the larger volumes of sample flowing through a membrane based system. Another method of separation can include microfluidic inertial focusing, where samples are passed through a small channel of a specific shape ratio causing separation of particles by size.

In addition to, or alternatively to, sample preparation, noise removal can also be employed. From a systems design perspective, this can be accomplished using lasers of a specific wavelength range to detect an increased, maximum, or threshold signal from organisms of a predefined size. For example, some microorganisms autofluoresce at particular wavelengths, for example around 600 nm. In a non-limiting embodiment, automatic power control (APC) lasers can maintain a sufficient and substantially constant intensity output without damaging a cell. A system that includes an APC laser could be enclosed in a light-proof box while ensuring dimensions and distances for high signal-to-noise ratio optical components such as filters, waveplates, beam splitters, etc. to reduce beam divergence.

In any of the embodiments of FIGS. 1-9, components can be selected for, optimized for, replaced with components to select for, etc. overall performance and/or cost, for example to address emerging growth markets. The signals obtained from any of the systems 100, 200, 300, 400, 500, 600, 700, 800, 900 are processed through one or more algorithms to identify one or more components or a signature indicating one or more components (e.g., chemicals, microorganisms, materials, etc.) of a sample.

FIG. 10 shows a schematic illustrating an embodiment of a method 1000 for sample identification including: analyzing an unknown sample with any of the systems 100, 200, 300, 400, 500, 600, 700, 800, 900 described herein at block 1010. The samples may be optically transparent and/or no lesser than about 0.1 um in dimension for increased performance. The samples can be illuminated using a light source (e.g., APC laser, diode laser, Gas laser, Diode Pumped Solid State Laser, fiber optics, etc.) to provide temperature stable power that can compensate for low signal from a small cross section and/or protect the sample from over illumination. For species such as mycobacteria, about 600 nm can also result in autofluorescence.

The method 1000 includes: scattered light from the unknown sample is measured at block 1020. Approximating laser wavelength with bacteria size increases scattering efficiency (Mie scattering). For example, for a bacteria of size range of about 650 nm, a system may illuminate the bacteria with similar wavelength (e.g., 650 nm or red). If the bacteria is illuminated with a longer wavelength, the light scattering may be isotropic, and not may not convey the information needed for identification. As such, in some embodiments, using a light source having a wavelength between about 300 nm to about 1000 nm may enable identification of bacteria. In some embodiments, using a light source having a wavelength between about 300 nm to about 1000 nm may also act as a natural filter, such that smaller and larger debris may be detected or processed by the system as background noise. This scattered polarized light may be physically filtered down to specific components such as those horizontally (H) or vertically (V) polarized, as described elsewhere herein. Filtering can use elements such as polarizers and waveplates selected to work in a wavelength range of a light source, as well as have high extinction ratios in a wavelength range to optimize signal-to-noise ratio.

The method 1000 includes identifying one or more patterns or signatures based on the scattered light (forward or back scattered) from the unknown sample at block 1030. Identifying can include computing quantities of interest such as Mueller matrix elements (i.e., scattering related to polarization state) or a Stokes four-vectors, and processing the data to extract quantities such as period, phase, amplitude, intensity (e.g., about polarization changes due to diattenuation, absorption, depolarization, etc.). In some embodiments, period, phase, and amplitude can also refer to properties of an angle resolved plot using elements of Mueller Matrix.

The method 1000 includes comparing (e.g., using principal component analysis, Fourier transforms, fast Fourier transforms, pattern recognition, feature analysis, etc.) the one or more patterns or signatures to a database or library including patterns or signatures from known samples at block 1040. The library is built using known samples (e.g., clinical and/or spiked samples already measured against a state-of-the-art comparator) that are measured and analyzed. The resulting data are stored as a library. In some embodiments, principal component analysis, fast Fourier transforms, and pattern recognition can be used in the comparing. In some embodiments, one or more of: a principal component analysis, a fast Fourier transform, or a pattern recognition can be used in the comparing.

The method 1000 includes determining whether the database includes a pattern or signature that substantially matches the pattern or signature for the unknown sample at block 1050. Block 1050 of method 1000 is described in further detail in connection with FIG. 11. The database may be queried using the cloud, for example where the library is remotely stored or the database may be queried locally, for example where the library is stored on any of the computing devices described herein or in any of the systems described herein. When yes (i.e., the signature for the unknown sample substantially matches a signature in the library) at block 1060, the method 1000 proceeds to identifying the unknown sample at block 1070. When no (i.e., the signature for the unknown sample does not substantially match a signature in the library) at block 1080, the method 1000 proceeds to updating the database or library with the pattern or signature. Updating the library or database can occur in real-time or can be a scheduled or latent synchronization. For some applications, such as door-to-door testing, part of the database may be stored on the device to make the system independent from internet access.

Turning to FIG. 11, which shows a method 1100 for sample identification. In some embodiments, prior to data processing, the system can be calibrated as described elsewhere herein and/or input data can be cleaned using background subtraction at block S1110. For example, a blank slide (or other optically transparent conduit or receptacle) may be inserted into any of the systems 100, 200, 300, 400, 500, 600, 700, 800, 900 described herein and signals (e.g., light scatter) resulting from the blank slide or receptacle can be measured. Light scatter can be detected for a sample of interest (i.e., actual sample) at block S1120 and the background subtracted from the detected light scatter from the sample of interest. The background signals and detected light scatter from the sample of interest may be detected, measured, and/or analyzed using any of system 100, 200, 300, 400, 500, 600, 700, 800, 900. Calibration and/or background correction enables measurement and collection of a variety of signatures regardless of a phase, concentration, media, etc. of any given species, etc. to facilitate the creation of a library of signatures. In field use, such a database can then be used to match signatures obtained from an unknown sample for real-time identification of species.

The method 1100 further includes identifying one or more signatures of the cleaned (i.e., background corrected) detected light scatter of the sample of interest at block S1140.

Identifying one or more signatures may include comparing the detected light scatter of the sample of interest to a library of signatures. The comparison may include one or more of: pattern recognition, principal component analysis (PCA) (at block S1160), Fourier and/or fast Fourier transformations (FFT) (at block S1150), feature analysis, as well as supervised learning and classification algorithms (at block S1170) such as nearest neighbor, random forest, etc. In some embodiments, the comparison may include a PCA. In some embodiments, the comparison may include an FFT. In some embodiments, the comparison may include pattern recognition. In some embodiments, the comparison may include analysis using a Neural Network. In some embodiments, one or more of the outputs from: the PCA, FFT, pattern recognition, or Neural Network may be compared, weighted, and/or combined to output an indication of the sample. In some embodiments, one or more of the outputs from: the PCA, FFT, pattern recognition, or Neural Network may be excluded from an overall output of an indication, for example based on an accuracy or quality of the output. For example, the indication may include a diagnostic result based on one or more identified optical signatures found in the sample. In some embodiments, the diagnostic result may include a phenotypic identification of at least one microorganism or one or more microorganisms. In some embodiments, the diagnostic result may include an indicator associated with a lack of a microorganism.

For example, any of the systems 100, 200, 300, 400, 500, 600, 700, 800, 900 can be trained for signatured determination and/or comparison of an unknown signature to a known signature using a trained neural network (convolutional neural network, Feedforward), as shown at block S1170. The neural network can be trained using known samples (e.g., clinical and/or spiked samples already measured against a state-of-the-art comparator) that are measured and analyzed. For example, determining whether a database includes a pattern or signature that substantially matches the pattern or signature for the unknown sample can be accomplished using machine learning predictions, as well as internal libraries to cross validate data obtained versus database and calculating area and distances between individual and summed components. In a non-limiting example, a machine learning model may be trained using supervised learning on labeled data. In some embodiments, the machine learning model is a feedforward type model with, for example, 10 hidden layers. Although 10 layers are described, one of skill in the art will appreciate that the number of layers is variable and may be adjusted. An indication of one or more signatures of the unknown sample or a component of the unknown sample may be output at block S1180. The output may be local, for example on a handheld device or a computing device communicatively coupled to the handheld device or remotely at a server or other remote computing device. For example, any of the computing devices of FIGS. 1-9.

In some embodiments, the detected light scatter for a sample of interest or unknown sample is processed using FFT, which can output phase and/or frequency for the sample of interest or unknown sample. If the output phase and/or frequency for the unknown sample falls into (or is within a predefined buffer, tolerance or threshold of) one or more regions of interest for various samples (e.g., known phase and/or frequency for known samples in the library), then the system may output an indication of a match or substantial match. In some embodiments, relevant features from the detected light scatter are extracted using a PCA. The extracted features can be fed into to a trained classifier model, which outputs one or more signatures. The signatures may be compared to a library of signatures to determine whether there is a partial or substantial match. In some embodiments, a model (e.g., Neural Network) may be trained using optical signatures of predetermined concentrations of known bacteria. In some embodiments, when optical signatures from an unknown sample are input into the trained model, the model outputs a predicted concentration and/or bacteria species.

Then when there's an unknown sample, the NN returns a predicted concentration. What we planned and are currently working on, is to do feature extraction-figuring out relevant features using PCA for the training set, and use that to get an output based on features. The easy way to do this without PCA is to come up with an ‘average’ curve for a species, figure out which segments of the curve are important and measure euclidean difference.

In some embodiments, the detected light scatter for a known sample is processed using PCA. For example, PCA can be performed to extract features from the optical signatures of the known sample that may be used to train a model. In some embodiments, extracted features from an optical signature of unknown sample are input into the trained model, which outputs a predicted bacteria species. In some embodiments, the detected light scatter for a sample of interest or an unknown sample is processed using PCA. For example, PCA can be performed on one or more known signatures (e.g., known scattered light from known samples in a library) and one or more unknown signatures (e.g., detected scattered light from an unknown sample) to determine which type of samples the unknown sample belongs to, how close the signature of the scattered light of unknown sample is to the center of a cluster, etc. Alternatively, to using PCA, an ‘average’ curve for one or more species may be determined. One or more segments of the average curve can be determined and the euclidean difference measured.

In some embodiments, a neural network can be used to predict a value of a concentration of one or more species based on pattern recognition. Any one or more of these analyses may be used independently or in combination to determine a signature of an unknown sample (or a signature of a component of an unknown sample) and output an indication of the signature at block S1180.

The systems and methods of the various embodiment and variations thereof can be embodied and/or implemented at least in part as a machine configured to receive a computer-readable medium storing computer-readable instructions. The instructions are preferably executed by computer-executable components preferably integrated with any of the systems described herein and one or more portions of the processor and/or computing device. The processor can be a central processing unit, a graphics processing unit, a field-programmable gate array, a digital signal processor, an application-specific integrated circuit, and the like. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

### EXAMPLES

Example 1. A portable system for detecting one or more microorganisms in a specimen, comprising: one or more light sources arranged to produce a light beam that is configured to interact with a receptacle positioned in the system; one or more filters, wherein the light beam is polarized by at least one of the one or more filters; one or more beam splitters; a plurality of sensors arranged along an arc, relative to a path of the light beam, and configured to detect scattered light at a plurality of angles relative to the receptacle; and a processor communicatively coupled to memory, the one or more light sources, and the plurality of sensors, the processor configured to execute instructions stored in the memory, the instructions comprising: receiving, from at least a subset of the plurality of sensors, detected scattered light signals from a sample that is positionable in the receptacle; identifying one or more optical signatures in the detected scatter signals from the sample, comparing the one or more identified optical signatures from the sample to a database of optical signatures, and outputting, based on the comparison, an indication of the one or more identified optical signatures from the sample.

Example 2. The portable system of any one of the preceding examples, but particularly Example 1, wherein: each of the one or more filters is positioned downstream of each of the one or more light sources; and each of the one or more beam splitters is positioned downstream of each of the one or more filters.

Example 3. The portable system of any one of the preceding examples, but particularly Example 1, wherein each of the one or more beam splitters is positioned downstream of each of the one or more light sources.

Example 4. The portable system of any one of the preceding examples, but particularly Example 1, wherein at least of the one or more lights sources, at least one of the one or more filters, and at least one of the one or more beam splitters are substantially longitudinally aligned along an x-axis.

Example 5. The portable system of any one of the preceding examples, but particularly Example 4, wherein the one or more light sources comprise a plurality of lights sources; the one or more filters comprise a plurality of filters; and the one or more beam splitters comprise a plurality of beam splitters.

Example 6. The portable system of any one of the preceding examples, but particularly Example 5, wherein a subset of the plurality of light sources, a subset of the plurality of filters, and a subset of the plurality of beam splitters are positioned orthogonally to the x-axis.

Example 7. The portable system of any one of the preceding examples, but particularly Example 4, wherein the plurality of sensors arranged along the arc is offset from the x-axis in an x-y plane by about 0 degrees to about 120 degrees.

Example 8. The portable system of any one of the preceding examples, but particularly Example 4, wherein the plurality of sensors arranged along the arc is offset from the x-axis in an x-y plane by about 0 degrees to about 90 degrees.

Example 9. The portable system of any one of the preceding examples, but particularly Example 1, wherein the receptacle is configured to receive a sample and position the sample between at least one of the one or more beam splitters and at least one of the plurality of sensors.

Example 10. The portable system of any one of the preceding examples, but particularly Example 1, further comprising the sample configured to be received within the receptacle.

Example 11. The portable system of any one of the preceding examples, but particularly Example 10, wherein the sample comprises one of: a blood sample, a serum sample, a sputum sample, a urine sample, a stool sample, a buccal swab sample, a throat swab sample, a nasal swab sample, or a saliva sample.

Example 12. The portable system of any one of the preceding examples, but particularly Example 1, wherein the one or more light sources comprise a monochromatic light source.

Example 13. The portable system of any one of the preceding examples, but particularly Example 1, wherein the one or more light sources comprise a laser.

Example 14. The portable system of any one of the preceding examples, but particularly Example 1, wherein the indication comprises a species of bacteria of the specimen.

Example 15. The portable system of any one of the preceding examples, but particularly Example 1, wherein the indication comprises a chemical component of the specimen.

Example 16. The portable system of any one of the preceding examples, but particularly Example 1, wherein the indication comprises a material component of the specimen.

Example 17. The portable system of any one of the preceding examples, but particularly Example 1, wherein identifying the one or more optical signatures in the detected scatter signals from the sample includes determining one or both of: one or more Mueller matrix parameters or one or more Stokes four-vectors parameters of the light beam.

Example 18. The portable system of any one of the preceding examples, but particularly Example 1, wherein comparing the one or more identified optical signatures from the sample to the database of optical signatures includes using one or more of: principal component analysis, fast Fourier Transforms, or a trained Neural Network to compare the one or more identified optical signatures to the database of optical signatures.

Example 19. The portable system of any one of the preceding examples, but particularly Example 1, wherein the instructions further comprise: calibrating the system by: receiving second scattered light signals, from a blank receptacle, from the one or more of the plurality of sensors; identifying one or more second optical signatures in the second scattered light signals from the blank receptacle; and subtracting the one or more second optical signatures from the blank receptacle from the one or more detected optical signatures from the sample.

Example 20. The portable system of any one of the preceding examples, but particularly Example 1, wherein the indication of the one or more identified optical signatures found in the sample comprises a diagnostic result for the unknown sample.

Example 21. The portable system of any one of the preceding examples, but particularly Example 20, wherein the diagnostic result comprises a phenotypic identification of at least one microorganism of the one or more of microorganisms.

Example 22. The portable system of any one of the preceding examples, but particularly Example 21, wherein the at least one microorganism is Mycobacterium Tuberculosis.

Example 23. The portable system of any one of the preceding examples, but particularly Example 20, wherein the diagnostic result comprises an indicator associated with a lack of the one or more of microorganisms.

Example 24. A portable system for detecting one or more microorganisms in a specimen, the system comprising: a light source arranged to produce a light beam that is configured to interact with a receptacle positioned in the system; a polarization state generator configured to polarize the light beam from the light source; a polarization state analyzer configured to receive the polarized light beam, the polarized light beam having interacted with a sample that is positionable in the receptacle, and determine one or both of: one or more Mueller matrix parameters or one or more Stokes four-vectors parameters of the polarized light beam; and one or more processors communicatively coupled to memory, the light source, the polarization generator, and the polarization state analyzer, the processor configured to receive instructions from the memory and execute the instructions comprising: receiving one or more determined parameters from the polarization state analyzer, the sample positionable in the receptacle, identifying one or more optical signatures of the sample based on the one or more determined parameters, comparing the one or more identified optical signatures of the sample to a database of signatures, and outputting an indication of the one or more identified optical signatures of the sample.

Example 25. The portable system of any one of the preceding examples, but particularly Example 24, wherein the polarization state analyzer is offset from the polarization state generator by about 0 degrees to about 120 degrees relative to a longitudinal axis of the polarization state generator.

Example 26. The portable system of any one of the preceding examples, but particularly Example 24, further comprising the receptacle configured to receive the sample and position the sample between the light source and the polarization state generator.

Example 27. The portable system of any one of the preceding examples, but particularly Example 26, further comprising the sample, wherein the sample comprises one of: a blood sample, a serum sample, a sputum sample, a urine sample, a stool sample, a buccal swab sample, a throat swab sample, a nasal swab sample, or a saliva sample.

Example 28. The portable system of any one of the preceding examples, but particularly Example 24, wherein the light source comprises a monochromatic light source.

Example 29. The portable system of any one of the preceding examples, but particularly Example 28, wherein the light source comprises a laser.

Example 30. The portable system of any one of the preceding examples, but particularly Example 24, wherein the receptacle is an optically transparent sample holder.

Example 31. The portable system of any one of the preceding examples, but particularly Example 24, wherein the indication of the one or more identified optical signatures found in the sample comprises a diagnostic result for the sample.

Example 32. The portable system of any one of the preceding examples, but particularly Example 31, wherein the diagnostic result comprises a phenotypic identification of at least one microorganism of the one or more of microorganisms.

Example 33. The portable system of any one of the preceding examples, but particularly Example 32, wherein the at least one microorganism is Mycobacterium Tuberculosis.

Example 34. The portable system of any one of the preceding examples, but particularly Example 31, wherein the diagnostic result comprises an indicator associated with a lack of the one or more of microorganisms.

Example 35. A computer-implemented method of identifying one or more microorganisms in a specimen, comprising: receiving detected scattered light signals, from one or more sensors, from a sample that is positionable in a receptacle, the sample having interacted with a polarized light beam; identifying one or more optical signatures in the detected scatter signals from the sample, comparing the one or more identified optical signatures from the sample to a database of optical signatures, and outputting, based on the comparison, an indication of the one or more identified optical signatures from the sample.

Example 36. The computer-implemented method of any one of the preceding examples, but particularly Example 35, wherein the sample comprises one of: a blood sample, a serum sample, a sputum sample, a urine sample, a stool sample, a buccal swab sample, a throat swab sample, a nasal swab sample, or a saliva sample.

Example 37. The computer-implemented method of any one of the preceding examples, but particularly Example 35, wherein identifying the one or more optical signatures in the detected scatter signals from the sample includes determining one or both of: one or more Mueller matrix parameters or one or more Stokes four-vectors parameters of the light beam.

Example 38. The computer-implemented method of any one of the preceding examples, but particularly Example 35, wherein comparing the one or more identified optical signatures from the sample to the database of optical signatures includes using one or more of: principal component analysis, fast Fourier Transforms, or a trained Neural Network to compare the one or more identified optical signatures to the database of optical signatures.

Example 39. The computer-implemented method of any one of the preceding examples, but particularly Example 35, further comprising: receiving second scattered light signals, from a blank receptacle, from the one or more sensors; identifying one or more second optical signatures in the second scattered light signals from the blank receptacle; and subtracting the one or more second optical signatures from the blank receptacle from the one or more detected optical signatures from the sample.

Example 40. The computer-implemented method of any one of the preceding examples, but particularly Example 35, wherein the indication of the one or more identified optical signatures found in the sample comprises a diagnostic result for the unknown sample.

Example 41. The computer-implemented method of any one of the preceding examples, but particularly Example 40, wherein the diagnostic result comprises a phenotypic identification of at least one microorganism of the one or more of microorganisms.

Example 42. The computer-implemented method of any one of the preceding examples, but particularly Example 41, wherein the at least one microorganism is Mycobacterium Tuberculosis.

Example 43. The computer-implemented method of any one of the preceding examples, but particularly Example 40, wherein the diagnostic result comprises an indicator associated with a lack of the one or more of microorganisms.

Example 44. A non-transitory computer-readable storage medium for use in conjunction with a computer, the computer-readable storage medium storing programs instructions that, when executed by the computer, cause the computer to carry out one or more operations comprising: receiving detected scattered light signals, from one or more sensors, from a sample that is positionable in a receptacle, the sample having interacted with a polarized light beam; identifying one or more optical signatures in the detected scatter signals from the sample, comparing the one or more identified optical signatures from the sample to a database of optical signatures, and outputting, based on the comparison, an indication of the one or more identified optical signatures from the sample.

Example 45. The non-transitory computer-readable storage medium of any one of the preceding examples, but particularly Example 44, wherein the sample comprises one of: a blood sample, a serum sample, a sputum sample, a urine sample, a stool sample, a buccal swab sample, a throat swab sample, a nasal swab sample, or a saliva sample.

Example 46. The non-transitory computer-readable storage medium of any one of the preceding examples, but particularly Example 44, wherein identifying the one or more optical signatures in the detected scatter signals from the sample includes determining one or both of: one or more Mueller matrix parameters or one or more Stokes four-vectors parameters of the light beam.

Example 47. The non-transitory computer-readable storage medium of any one of the preceding examples, but particularly Example 44, wherein comparing the one or more identified optical signatures from the sample to the database of optical signatures includes using one or more of: principal component analysis, fast Fourier Transforms, or a trained Neural Network to compare the one or more identified optical signatures to the database of optical signatures.

Example 48. The non-transitory computer-readable storage medium of any one of the preceding examples, but particularly Example 44, wherein the one or more operations further comprise: receiving second scattered light signals, from a blank receptacle, from the one or more sensors; identifying one or more second optical signatures in the second scattered light signals from the blank receptacle; and subtracting the one or more second optical signatures from the blank receptacle from the one or more detected optical signatures from the sample.

Example 49. The non-transitory computer-readable storage medium of any one of the preceding examples, but particularly Example 44, wherein the indication of the one or more identified optical signatures found in the sample comprises a diagnostic result for the unknown sample.

Example 50. The non-transitory computer-readable storage medium of any one of the preceding examples, but particularly Example 49, wherein the diagnostic result comprises a phenotypic identification of at least one microorganism of the one or more of microorganisms.

Example 51. The non-transitory computer-readable storage medium of any one of the preceding examples, but particularly Example 50, wherein the at least one microorganism is Mycobacterium Tuberculosis.

Example 52. The non-transitory computer-readable storage medium of any one of the preceding examples, but particularly Example 49, wherein the diagnostic result comprises an indicator associated with a lack of the one or more of microorganisms.

As used in the description and claims, the singular form “a”, “an” and “the” include both singular and plural references unless the context clearly dictates otherwise. For example, the term “algorithm” may include, and is contemplated to include, a plurality of algorithms. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A portable system for detecting one or more microorganisms in a specimen, comprising:
one or more light sources arranged to produce a light beam that is configured to interact with a receptacle positioned in the system;
one or more filters, wherein the light beam is polarized by at least one of the one or more filters;
one or more beam splitters;
a plurality of sensors arranged along an arc, relative to a path of the light beam, and configured to detect scattered light at a plurality of angles relative to the receptacle; and
a processor communicatively coupled to memory, the one or more light sources, and the plurality of sensors, the processor configured to execute instructions stored in the memory, the instructions comprising:
receiving, from at least a subset of the plurality of sensors, detected scattered light signals from a sample that is positionable in the receptacle;
identifying one or more optical signatures in the detected scatter signals from the sample,
comparing the one or more identified optical signatures from the sample to a database of optical signatures, and
outputting, based on the comparison, an indication of the one or more identified optical signatures from the sample.

2. The portable system of claim 1, wherein: each of the one or more filters is positioned downstream of each of the one or more light sources; and each of the one or more beam splitters is positioned downstream of each of the one or more filters.

3. The portable system of claim 1, wherein each of the one or more beam splitters is positioned downstream of each of the one or more light sources.

4. The portable system of claim 1, wherein at least of the one or more lights sources, at least one of the one or more filters, and at least one of the one or more beam splitters are substantially longitudinally aligned along an x-axis.

5. The portable system of claim 4, wherein the one or more light sources comprise a plurality of lights sources; the one or more filters comprise a plurality of filters; and the one or more beam splitters comprise a plurality of beam splitters.

6. The portable system of claim 5, wherein a subset of the plurality of light sources, a subset of the plurality of filters, and a subset of the plurality of beam splitters are positioned orthogonally to the x-axis.

7. The portable system of claim 4, wherein the plurality of sensors arranged along the arc is offset from the x-axis in an x-y plane by about 0 degrees to about 120 degrees.

8. The portable system of claim 4, wherein the plurality of sensors arranged along the arc is offset from the x-axis in an x-y plane by about 0 degrees to about 90 degrees.

9. The portable system of claim 1, further comprising the receptacle, wherein the receptacle is configured to receive a sample and position the sample between at least one of the one or more beam splitters and at least one of the plurality of sensors.

10. The portable system of claim 1, further comprising the sample configured to be received within the receptacle.

11. The portable system of claim 10, wherein the sample comprises one of: a blood sample, a serum sample, a sputum sample, a urine sample, a stool sample, a buccal swab sample, a throat swab sample, a nasal swab sample, or a saliva sample.

12. The portable system of claim 1, wherein the one or more light sources comprise a monochromatic light source.

13. The portable system of claim 1, wherein the one or more light sources comprise a laser.

14. The portable system of claim 1, wherein the indication comprises a species of bacteria of the specimen.

15. The portable system of claim 1, wherein the indication comprises a chemical component of the specimen.

16. The portable system of claim 1, wherein the indication comprises a material component of the specimen.

17. The portable system of claim 1, wherein identifying the one or more optical signatures in the detected scatter signals from the sample includes determining one or both of: one or more Mueller matrix parameters or one or more Stokes four-vectors parameters of the light beam.

18. The portable system of claim 1, wherein comparing the one or more identified optical signatures from the sample to the database of optical signatures includes using one or more of: principal component analysis, fast Fourier Transforms, or a trained Neural Network to compare the one or more identified optical signatures to the database of optical signatures.

19. The portable system of claim 1, wherein the instructions further comprise:
calibrating the system by:
receiving second scattered light signals, from a blank receptacle, from the one or more of the plurality of sensors;
identifying one or more second optical signatures in the second scattered light signals from the blank receptacle; and
subtracting the one or more second optical signatures from the blank receptacle from the one or more detected optical signatures from the sample.

20. The portable system of claim 1, wherein the indication of the one or more identified optical signatures found in the sample comprises a diagnostic result, wherein the diagnostic results comprises a phenotypic identification of at least one microorganism of the one or more of microorganisms in the sample.

21.-52. (canceled)
